(19) United States
(12) Patent Application Publication
(10) Pub. No.: US 2006/0004214 A1
(43) Pub. Date: Jan. 5, 2006

(54) Methods for preparing 1,1,1-tris(4-hydroxyphenyl)alkanes

(76) Inventors: Gurram Kishan, Bangalore (IN); G.V. Ramanarayanan, Bangalore (IN); Khanwelkar Rahul Ravindra, Thane (IN); Adil Minoo Dhalla, Mumbai (IN); Vinod Kumar Rai, Bangalore (IN); Surendra Umesh Kulkarni, Bangalore (IN)

Correspondence Address: CANTOR COLBURN LLP, 55 GRIFFIN RD SOUTH, BLOOMFIELD, CT, 06002, US

(21) Appl. No.: 10/882,812

(22) Filed: Jun. 30, 2004

(51) Int. Cl. C07C 37/14

(52) U.S. Cl. 552/101

(57) ABSTRACT

A method for preparing 1,1,1,-tris(4-hydroxyphenyl)alkanes generally comprises reacting a mixture of an aromatic hydroxy compound and a ketone in the presence of at least one sulfonic acid catalyst and a mercaptan co-catalyst to produce the 1,1,1-tris(4-hydroxyphenyl)alkanes of formula:

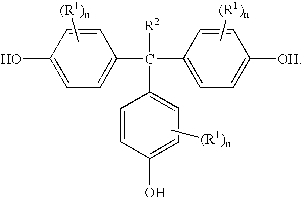

### BACKGROUND

The present disclosure generally relates to a method for preparing 1,1,1-tris(4-hydroxyphenyl)alkanes.

The 1,1,1-tris(4-hydroxyphenyl)alkanes, such as, for example, those disclosed in U.S. Pat. Nos. 3,579,542 and 4,992,598, can be used as branching agents during the polymerization of polycarbonates, for example. As such, it may be incorporated into reaction mixtures containing dihydroxy aromatic compounds such as bisphenol A and carbonate sources such as phosgene or diphenyl carbonate, among others.

An exemplary 1,1,1-tris(4-hydroxyphenyl)alkane, 1,1,1,-tris(4-hydroxphenyl)ethane (also referred to as THPE), can generally be prepared by the reaction of 4-hydroxyacetophenone with phenol. The reaction is analogous to the well known reaction of phenol with acetone to form 2,2-bis(4-hydroxyphenyl)propane (also commonly referred to as “bisphenol A”).

Alternate methods to prepare 1,1,1-tris(4-hydroxyphenyl)alkanes, such as THPE, include the reaction of 2,4-pentanedione with phenol in the presence of relatively volatile acids, e.g., gaseous hydrochloric acid, and a promoter such as a mercaptocarboxylic acid. Among the disadvantages to this method are that the quantity of catalyst used is relatively high and the volatile acids employed, e.g., hydrogen chloride gas, are generally corrosive. Other methods include the use of sulfuric acid in conjunction with 3-mercaptosulfonic acid as the promoter.

Accordingly, there remains a need in the art of methods for preparing 1,1,1-tris(4-hydroxyphenyl)alkanes in high yields, which use less expensive promoters that are easy to handle and/or catalyst materials that can be recycled.

### BRIEF SUMMARY

Disclosed herein are methods for producing 1,1,1-tris(4-hydroxyphenyl)alkanes. In one embodiment, the method comprises reacting a mixture of an aromatic hydroxy compound and a ketone in the presence of at least one sulfonic acid catalyst and a mercaptan co-catalyst to produce the 1,1,1-tris(4-hydroxyphenyl)alkane

In another embodiment, the method comprises reacting a mixture comprising an aromatic hydroxy compound and a ketone in the presence of at least one sulfonic acid catalyst and a mercaptan co-catalyst; contacting the mixture with a solvent to precipitate and isolate a filtrate and a 1,1,1-tris(4-hydroxyphenyl)alkane of formula: 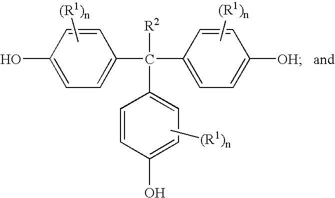
selectively removing the solvent from the filtrate to obtain a residue comprising the sulfonic acid catalyst and the mercaptan co-catalyst. The method may further comprise reacting the aromatic hydroxy compound and the ketone in the presence of the residue to form the 1,1,1-tris(4-hydroxyphenyl)alkane.

A method for forming 1,1,1-tris(4-hydroxyphenyl)ethane comprises reacting a mixture of a phenol and a 2,4-pentanedione in the presence of at least one sulfonic acid catalyst and a mercaptan co-catalyst to form the 1,1,1-tris(4-hydroxyphenyl)ethane.

In another embodiment, the method for forming 1,1,1-tris(4-hydroxyphenyl)ethane comprises reacting a mixture of phenol and 4-hydroxyacetophenone in the presence of at least one sulfonic acid catalyst and a mercaptan co-catalyst to form the 1,1,1-tris(4-hydroxyphenyl)ethane.

The above-described method may be understood more readily by reference to the following detailed description of the various features of the disclosure and the examples included therein.

### DETAILED DESCRIPTION

Disclosed herein are processes for preparing 1,1,1-tris(4-hydroxyphenyl)alkanes such as those represented by Formula (I), 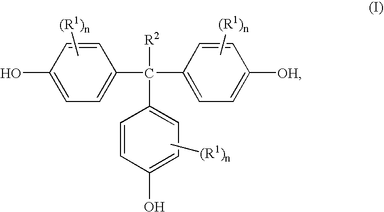
wherein R¹ and R² are independently at each occurrence a hydrocarbyl group and n is an integer of value 0-3. These compounds can be used as branching agents in the preparation of polymers, for example.

Representative hydrocarbyls that may be considered as the R¹ and R² groups are alkyl groups having 1 to 25 carbon atoms, such as, for example, methyl, ethyl, propyl, butyl, pentyl, hexyl, heptyl, octyl, nonyl, undecyl, decyl, dodecyl, and the isomeric forms thereof; aryl groups having 6 to 25 carbon atoms, such as ring-substituted and ring-unsubstituted forms of phenyl, tolyl, xylyl, naphthyl, biphenyl, tetraphenyl, and the like; aralkyl groups having 7 to 25 carbon atoms, such as ring-substituted and ring-unsubstituted forms of benzyl, phenethyl, phenpropyl, phenbutyl, naphthoctyl, and the like; and cycloalkyl groups, such as cyclopropyl, cyclobutyl, cyclopentyl, cyclohexyl, cycloheptyl, cyclooctyl, and the like.

As used herein, the singular forms “a”, “an” and “the” include plural referents unless the context clearly dictates otherwise.

“Optional” or “optionally” means that the subsequently described event or circumstance may or may not occur, and that the description includes instances where the event occurs and instances where it does not.

In accordance with the present disclosure, the process for preparing the 1,1,1-tris(4-hydroxyphenyl)alkanes generally comprises reacting an aromatic hydroxy compound of Formula (II), 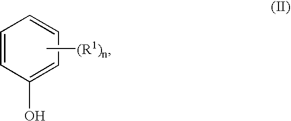
with a ketone of Formula (III) and/or Formula (IV), 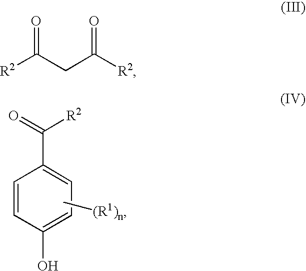
wherein R¹, R², and “n” are as defined above. The aromatic hydroxy compound and the ketone can be reacted in the presence of at least one sulfonic acid catalyst and a mercaptan co-catalyst.

In one embodiment, the aromatic hydroxy compound may be selected from the group consisting of substituted or unsubstituted phenols. Examples of suitable aromatic hydroxy compounds include, but are not limited to, 2,6-dimethylphenol, 2,3,6-trimethylphenol, 2,6-di-tert-butylphenol, 2-tert-butylphenol, meta-cresol, ortho-cresol, ortho-phenylphenol, ortho-benzylphenol, and mixtures of the foregoing aromatic hydroxy compounds. In one particular embodiment, the aromatic hydroxy compound is phenol.

Exemplary ketones of Formula (III) include, but are not intended to be limited to, 2,4-pentanedione and exemplary ketones of Formula IV include, but are not intended to be limited to, 4-hydroxyacetophenone. These particular ketones, while not intended to be limiting, can generally be utilized because of their commercial availability and low cost, among others. In one embodiment, the 1,1,1-tris(4-hydroxyphenyl)alkane prepared by the method of this disclosure is 1,1,1-tris(4-hydroxyphenyl)ethane (THPE).

The molar ratio of the aromatic hydroxy compound to the ketone is 5-30 to 1. In one embodiment, the molar ratio of the aromatic hydroxy compound to the ketone is 8-15 to 1. In other embodiments, the molar ratio of the aromatic hydroxy compound to ketone is 10-13 to 1. The reactants comprising the aromatic hydroxy compound, the ketone, the mercaptan co-catalyst and the acid catalyst can be blended in any order. In one embodiment, the ketone is introduced last and incrementally (e.g. drop-wise) in order to maintain an excess of phenol during the reaction. In another embodiment, the acid catalyst is introduced last and incrementally (e.g. drop-wise), to improve product selectivity.

The sulfonic acid catalyst generally comprises, straight or branched chain aliphatic sulfonic acids or aromatic sulfonic acids having 1 to 20 carbon atoms. More than one sulfonic acid catalyst may be present. Non-limiting examples of these sulfonic acids include methanesulfonic acid, ethanesulfonic acid, propanesulfonic acid, butanesulfonic acid, naphthalenesulfonic acid, p-toluenesulfonic acid, and dodecylbenzenesulfonic acid. In one embodiment, the acid catalyst employed is methanesulfonic acid. The proportion of the sulfonic acid catalyst in the reaction mixture, calculated as the free acid, is 1 to 20 weight percent based on total weight of the reaction mixture. More specifically, the proportion is 3 to 15 weight percent and most specifically the proportion is 4 to 8 weight percent based on total weight of the reaction mixture.

The co-catalyst generally comprises a mercaptan compound of Formula (V),
[B [symbol] ₘ [symbol] A [symbol] [symbol] C] (V),
wherein A is a monovalent or divalent hydrocarbyl group having 1 to 12 carbon atoms; B is selected from the group consisting of an hydrogen, a hydroxyl, —S—H, —S—R³, —COOR⁴ and SO₃R⁴; and C is selected from the group consisting of —S—H, —S—R³, —SCOOR⁴ and SCOR⁴, wherein R³ is a tertiary alkyl group having 4 to 25 carbon atoms and R⁴ is selected from the group consisting of a hydrogen and a monovalent hydrocarbyl group having 1 to 12 carbon atoms, and m is an integer having a value 0 or 1.

Representative hydrocarbyls that may be considered as the A and the R⁴ groups may be straight chain or branched chain alkyl groups having 1 to 12 carbon atoms, such as, for example, methyl, ethyl, propyl, butyl, pentyl, hexyl, heptyl, octyl, nonyl, undecyl, decyl, dodecyl, and the isomeric forms thereof; aryl groups having 6 to 12 carbon atoms, such as ring-substituted and ring-unsubstituted forms of phenyl, tolyl, xylyl, naphthyl, biphenyl and the like; aralkyl groups having 7 to 12 carbon atoms, such as ring-substituted and ring-unsubstituted forms of benzyl, phenethyl, phenpropyl, phenbutyl, and the like; and cycloalkyl groups, such as cyclopropyl, cyclobutyl, cyclopentyl, cyclohexyl, cycloheptyl, cyclooctyl, and the like.

Optionally, the mercaptan co-catalyst can be employed in salt form. If the co-catalyst employed is in the salt form, the at least one sulfonic acid is used in a stoichiometric excess to obtain the free acid of the co-catalyst in situ. For example, a sodium salt of 3-mercaptopropionic acid can be converted to 3-mercaptopropionic acid upon contact with the acid catalyst.

Non-limiting examples of suitable mercaptan co-catalysts include 3-mercaptopropionic acid (hereinafter called 3-MPA), a substituted or an unsubstituted benzyl mercaptan, 3-mercapto-1-propanol, ethyl 3-mercaptopropionate, 1,4-bis(mercaptomethyl)benzene, 2-mercaptoethane-sulfonic acid, 3-mercaptopropanesulfonic acid, 4-mercaptobutanesulfonic acid, 4-mercaptopentane-sulfonic acid, 3-mercapto-2,2-dimethylpropanesulfonic acid, 2,3-dimercaptopropanesulfonic acid, mercaptopropane-2,3-disulfonic acid, 2-benzyl-4-mercaptobutanesulfonic acid, 5-mercaptopentane-sulfonic acid, methanethiol, ethanethiol, isopropanethiol, butanethiol and mixtures of the foregoing mercaptan co-catalysts. In one embodiment, 3-mercaptopropionic acid can be utilized because of its commercial availability and low cost, among others.

The quantity of mercaptan co-catalyst employed in the reaction is 0.01 weight percent to 10 weight percent based on total weight of the reaction mixture. In other embodiments, the quantity of mercaptan co-catalyst employed is 0.05 weight percent to 5 weight percent based on total weight of the reaction mixture. In still other embodiments, the quantity of mercaptan co-catalyst employed is 0.75 weight percent to 3 weight percent based on total weight of the reaction mixture.

The reaction may be carried out at a temperature of 30° C. to 100° C. and more specifically at 40° C. to 80° C. In one embodiment, the reaction temperature is of 45° C. to 60° C. Although temperatures less than 30° C. may be used, the reaction rate is relatively slow and may not be desirable for some applications. At temperatures above 70° C., competing reactions involving the ketone can occur, which may decrease the yield of 1,1,1-tris(4-hydroxyphenyl)alkane and as such, may not be desirable for some applications. The time taken for the reaction varies from 10 hours to 50 hours. In other embodiments, the reaction time varies from 15 to 40 hours and in still other embodiments, the reaction time varies from 20 hours to 30 hours. Optionally, the reaction may be carried out in an inert atmosphere such as in the presence of nitrogen, helium or argon.

The reaction mixture so-obtained can then be contacted with a solvent to precipitate a solid material, e.g., the 1,1,1-tris (4-hydroxyphenyl)alkane. The solvent used for the precipitation can be, but is not limited to, chlorinated solvents, toluene, xylene or mixtures of the foregoing solvents thereof. Non-limiting examples of suitable chlorinated solvents include methylene chloride, ethylene dichloride, dichlorobenzene and chlorobenzene. In one embodiment, the solvent used is methylene chloride. Generally, the amount of solvent used comprises a volume ratio to the reaction mixture of at least 2:1, more specifically the volume ratio is 2.5:1, and most specifically the volume ratio is 3:1. More solvent may be used, but this may lead to a decrease in the yields of 1,1,1-tris (4-hydroxyphenyl)alkane. The pale yellowish brown solid material that precipitates contains at least 90% by weight of the particular 1,1,1-tris (4-hydroxyphenyl)alkane.

The solid material may subsequently be contacted with a methanol-water mixture containing at least 20% methanol by volume for 0.5 to 2 hours. In one embodiment, the proportion of methanol in the methanol-water mixture is on the order of 20-40% by volume. The solid material may then be further refluxed in a mixture comprising an alcohol containing a decolorizing agent. Suitable decolorizing agents include, but are not intended to be limited to, alkali metal borohydrides, alkali metal dithionites, activated charcoal, combinations comprising at least one of the foregoing decolorizers, and the like. Suitable alcohols comprise straight chain or branched or cyclic aliphatic alcohols containing from 1 to 8 carbon atoms. Non-limiting examples of suitable aliphatic alcohols include methanol, ethanol, iso-propanol, iso-butanol, n-butanol, tertiary-butanol, n-pentanol, iso-pentanol, mixtures of at least one of the foregoing aliphatic alcohols, and the like. The mixture may optionally be treated with activated charcoal to achieve further decolorization if desired. The mixture can then be treated with water, optionally containing the decolorizing agent, to precipitate a visually colorless 1,1,1-tris(4-hydroxyphenyl)alkane, for example.

By way of illustration, the reaction mixture produced when the aromatic hydroxy compound is phenol and the ketone is 2,4-pentanedione, comprises a mixture of THPE, bisphenol-A, and unreacted starting compounds. When the ketone used is 4-hydroxyacetophenone, the resultant reaction mixture comprises THPE and unreacted starting compounds, wherein no bisphenol-A is produced.

In another embodiment, the sulfonic acid catalyst and the solvent employed in the reaction may be recovered and recycled. The solvents are generally recovered by a distillation process while maintaining the temperature of the reaction mixture at 30° C. to 80° C., under vacuum. The residue obtained upon removal of the solvent generally comprises the sulfonic acid catalyst and the unreacted aromatic hydroxy compound, which can advantageously be recycled in the next reaction. The residue can then be mixed with make-up quantities of the aromatic hydroxy compound, the ketone, the mercaptan co-catalyst, and the acid catalyst. The reaction using the recycled residue proceeds under similar reaction conditions as discussed hereinabove and can be used to provide a purified 1,1,1-tris(4-hydroxyphenyl)alkane product.

As previously discussed, the 1,1,1-tris(4-hydroxyphenyl)alkanes obtained herein can be used as branching agents such as may be desired for producing—branched polycarbonates. For example, THPE can be added to the reactants used during polymerization. The desired rheological effects of branching provide higher viscosities and higher melt strengths relative to an otherwise similar resin prepared without using THPE. Branched polycarbonates derived from 1,1,1-tris(4-hydroxyphenyl)alkane are suitable for use as films or sheets. The branched polycarbonates can also be blow molded to prepare structured containers.

A number of polymerization methods can be used for producing the branched polycarbonates, comprising the 1,1,1-tris(4-hydroxyphenyl)alkanes. Suitable methods for fabricating these polycarbonates, for example, include a melt transesterification polymerization method and an interfacial polymerization method.

The melt transesterification polymerization method is generally carried out by combining a catalyst (e.g., quaternary phosphonium salts or sodium hydroxide or tetraalkylammonium salts) and a reactant composition to form a reaction mixture. Next the reaction mixture is mixed under sufficient pressure and temperature conditions for a time period effective to produce a branched polycarbonate. The resultant product mixture generally comprises a carbonic acid diester of the formula (ZO)₂C═O, wherein each Z is independently an unsubstituted or a substituted alkyl radical, or an unsubstituted or a substituted aryl radical and the 1,1,1-tris(4-hydroxyphenyl)alkane.

In the interfacial polymerization method, 1,1,1-tris(4-hydroxyphenyl)alkane, one or more comonomers, and phosgene are reacted in the presence of an acid acceptor and an aqueous base to produce a polycarbonate. Tertiary amines, such as for example, trialkylamines are preferably used as acid acceptors. An exemplary trialkylamine is triethylamine. Suitable aqueous bases include, for example, the alkali metal hydroxides, such as sodium hydroxide.

The following examples fall within the scope of, and serve to exemplify, the more generally described methods set forth above. The examples are presented for illustrative purposes only, and are not intended to limit the scope of the invention.

In the following examples, a high performance liquid chromatography (HPLC) method was used to quantify the conversion of the aromatic hydroxy compound of Formula (II) and the ketone of Formula (III) and/or Formula (IV) to the particular 1,1,1-tris(4-hydroxyphenyl)alkane. The HPLC was initially calibrated using standard samples of aromatic hydroxy compound, ketone, 1,1,1-tris(4-hydroxyphenyl)alkane, and bisphenol. The standard samples were diluted with acetonitrile and injected into a Zorbax XDB, C8 5μ reverse phase column commercially available from Agilent Technologies. Each reaction mixture was then diluted with acetonitrile and a sample of which was injected into a Zorbax XDB, C8 5μ column. Samples at specific time intervals were analyzed and compared to the HPLC chromatogram of the standard samples to follow the formation of 1,1,1-tris(4-hydroxyphenyl) alkane in the reaction.

The color value of the 1,1,1-tris(4-hydroxyphenyl)alkanes prepared by following the methods of this disclosure preferably have a percentage transmission at the corresponding wavelengths as indicated in Table 1 below

TABLE 1

| Wavelength | |
|---|---|
| (nanometers) | % Transmission not less than |
| 440 | 50 |
| 560 | 80 |
| 630 | 80 |

### EXAMPLE 1

In this example, THPE was prepared from 4-hydroxyacetophenone, phenol, mercaptopropionic acid, and methane sulfonic acid. Phenol (200 grams (g)) was charged into a 500 milliliters (ml) 4-necked round bottom flask equipped with a mechanical stirrer, thermometer pocket, and a water-cooled reflux condenser with a calcium chloride guard and an air leak tube. The flask was then heated to 55° C. and maintained under nitrogen atmosphere, while stirring. Next, p-hydroxyacetophenone (34 g) and 3-mercaptopropionic acid (5.5 g) were added. The methane sulfonic acid (14.81 g) was then added in a drop wise manner over about a thirty minute period. The reaction mixture was maintained at 55° C. under nitrogen atmosphere for 20 hours. The reaction mixture was then cooled room temperature (RT, 24° C.) and the nitrogen flow was stopped. The reactants of the flask were transferred into a 1 liter (L) beaker containing ethylene dichloride (600 ml) and stirred for 2 hours. The solids were filtered to get a crude product weighing approximately 58 g. The crude product was then subjected to a purification process as described below.

The purification process included stirring the crude reaction product into a methanol-water mixture (40:60 volume be volume, 120 ml) for 0.5 hours. Next the solids were filtered off, and the process was repeated with additional methanol-water (80 ml). The solids so obtained were then dissolved in methanol (120 ml). Sodium borohydride (NaBH₄, 150 milligrams) was added to this mixture, followed by stirring for half an hour. The solution was then treated with 1 gram of charcoal and subsequently filtered. 280 ml of water containing sodium borohydride (0.0125% weight by volume) was added to the filtrate over a period of 2 hours under nitrogen atmosphere. Another 120 ml of water containing 0.0125% w/v of sodium borohydride was added to this mixture all at once and stirred for 2 hours. The solids were then filtered and washed with 100 ml of 20% volume by volume of methanol in water. The purified solids were dried at 60° C. under vacuum to constant weight to provide 53 g of product.

The ethylene dichloride used to isolate the solids was recovered by distilling the reaction mixture while maintaining the reaction temperature at 50-55° C., under vacuum, initially at 180 mm and at the end at 60 mm. The residue so obtained was recycled in the next batch.

### EXAMPLE 2

In this example, the residue obtained in Example 1 was recycled.

Recycle 2a: The residue was used in the next batch with phenol (46.4 g), p-hydroxyacetophenone (34 g), 3-mercaptopropionic acid (2.44 g) and methane sulfonic acid (8.9 g). The reaction was carried out in a similar manner as the original batch to get a purified THPE (58.98 g).

Recycle 2b: The residue obtained from the filtrate of recycle 1a was reacted in a similar manner with phenol (59 g), p-hydroxyacetophenone (34 g), 3-mercaptopropionic acid (2.44 g) and methane sulfonic acid (8.9 g) to provide a purified THPE (56.03 g).

Unreacted phenol was obtained by distilling the filtrate under vacuum (distillation temperature 61-62° C. at 0.4 mm of Hg). The results of Example 1, and the recycle steps are tabulated in Table 2 below.

### EXAMPLE 3

In this example, THPE was prepared in accordance with Example 1 using the phenol recovered from Example 1. The results are tabulated in Table 2 below.

TABLE 2

| | Raw materials in grams | | | | Purified yields | |
|---|---|---|---|---|---|---|
| Example | phenol | 4-HAP | 3-MPA | MSA | Grams | % |
| 1 | 200 | 34 | 5.5 | 14.8 | 44.19 | 57.76 |
| Recycle 2a | 46.4 | 34 | 2.44 | 8.89 | 58.98 | 77.09 |
| Recycle 2b | 59 | 34 | 2.44 | 8.89 | 56.03 | 73.24 |
| 3 | 160* | 25.5 | 4.15 | 11.12 | 33.77 | 58.85 |

*indicates recycled phenol was used in the reaction

### EXAMPLE 4

In this example, THPE was prepared using phenol and 2,4-pentanedione.

Phenol (306 g) 2,4-Pentanedione (PD, 25 g) and 3-mercaptopropionic acid (3-MPA, 4.96 g) were charged to a 1 L 4-necked round bottom flask equipped with overhead stirrer and a nitrogen inlet. To this reaction mixture methanesulfonic acid (MSA, 16.5 g) was added in a drop-wise manner maintaining the temperature below 35° C. After complete addition of MSA, the temperature was raised to 55° C. and stirring was continued for 22 hours. The reaction mixture turned into a thick red slurry. This red slurry was poured into 2 L conical flask and diluted with 1.1 L of ethylene dichloride (EDC) and stirred for 3 hours at room temperature (RT, 24° C.). The solid material that precipitated from this reaction mixture was then filtered and washed with sufficient amount of EDC (approximately 150 ml) till the filtrate obtained was colorless. The buff colored crude solid obtained was dried on rotavac for 3 hours to 4 hours (at 45° C., 50 millibar). The solids were taken in a 1 L round bottom flask and slurried with 150 ml. of methanol:water (20:80 volume by volume) and stirred for half an hour. The solid material obtained was filtered and dried in rotavac under vacuum for 3 hours to 4 hours (at 60° C., 50 millibar). The solids so obtained were then refluxed in 85 ml. of methanol and 50 milligrams (mg) of NaBH₄ was added. The color of the solution turned to pale yellow to which 170 ml of demineralized water containing 50 mg of NaBH₄ was added. This mixture was then allowed to cool to room temperature and stirred for 2 hours. Solid material precipitated out. This was then filtered and washed with approximately 50 ml cold methanol:water (20:80) till the filtrate obtained colorless. The solid material so obtained was dried on rotavac for 3 hours to 4 hours (at 60° C., 50 millibar) to provide a white colored product with combined THPE and BPA together purity >99.5%, by HPLC and color test (UV test) in about 52% yield.

The color values were analyzed spectrophotometrically by weighing 2.5 grams of the particular 1,1,1-tris(4-hydroxyphenyl)alkane in a 30 milliliters vial and dissolving the sample in 5 grams of methanol. The percentage transmission was then measured using this solution in a UV-visible spectrophotometer in the range of 300 nanometer (nm) to 750 nm.

### EXAMPLES 5-11

These examples were carried out in a similar manner as described in Example 4, and the recycle steps were carried out as in Example 1. The results are shown in Table 3 below. Table 3 indicates the transmission values in percentage in the UV visible range for some of the Examples 4-11 in addition to providing the yields obtained in these examples.

TABLE 3

| | Raw | | | | Crude | | Final | | UV | | |
|---|---|---|---|---|---|---|---|---|---|---|---|
| | materials | | | | yields | | yields | | results | | |
| Example | phenol | PD | 3-MPA | MSA | g | % | g | % | 440 nm | 560 nm | 630 nm |
| 4 | 306 | 25 | 4.96 | 16.5 | 42.5 | 55 | 40 | 52.3 | NA | NA | NA |
| 5 | 306 | 25 | 4.96 | 16.5 | 40.8 | 53.3 | 38 | 50 | 64.76 | 82.88 | 89.51 |
| 6 | 306 | 25 | 4.96 | 16.5 | 40.8 | 53.3 | 38 | 50 | 75.1 | 86.7 | 90.51 |
| 7 | 376 | 26.88 | 6.47 | 20.5 | 48.7 | 59.3 | 42 | 51.2 | NA | NA | NA |
| 8 | 306 | 25 | 4.96 | 16.5 | 40.76 | 53.28 | 37.5 | 49 | NA | NA | NA |
| 9 | 306 | 25 | 4.96 | 16.5 | 42 | 55 | 39.5 | 51.6 | NA | NA | NA |
| Recycle 9 | 118 | 25 | 2.48 | 8.27 | 36 | 47 | 32.6 | 42.7 | 59.3 | 81.4 | 87.1 |
| 10 | 200 | 25 | 3.375 | 11.25 | 33.5 | 43.8 | 31.7 | 41.4 | NA | NA | NA |
| 11 | 235 | 25 | 3.5 | 11.76 | 35.58 | 46.5 | 33.12 | 43.3 | 57.5 | 81 | 90.7 |
| Recycle 11a | 118 | 25 | 1.75 | 5.88 | 32.3 | 42.2 | 27.2 | 35.6 | 83.8 | 94.3 | 96.1 |
| Recycle 11b | 118 | 25 | 1.75 | 5.88 | 33.8 | 44.18 | 26.06 | 34.1 | 76.9 | 90.4 | 93.9 |

NA—Not Available

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. The disclosure is further illustrated by the following non-limiting examples.

1. A method comprising:
reacting a mixture comprising an aromatic hydroxy compound and a ketone in the presence of at least one organic sulfonic acid catalyst and a mercaptan co-catalyst to produce a 1,1,1-tris(4-hydroxyphenyl)alkane of formula: 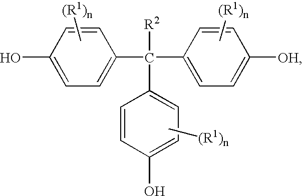
wherein the aromatic hydroxy compound has a formula of: 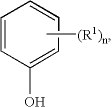
and wherein the ketone has a formula of: 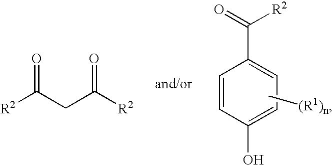
wherein R¹ and R² are independently at each occurrence a hydrocarbyl group and n is an integer of value 0-3, wherein the organic sulfonic acid is in an amount from 4 to 8 weight percent based on a total weight of the mixture and wherein the mercaptan co-catalyst is in an amount from 0.75 weight percent to 3 weight percent based on the total weight of the mixture.

2. The method of claim 1, wherein the 1,1,1-tris(4-hydroxyphenyl)alkane is 1,1,1-tris (4-hydroxyphenyl)ethane.

3. The method of claim 1, wherein the aromatic hydroxy compound is phenol.

4. The method of claim 1, wherein the ketone is 2,4-pentanedione.

5. The method of claim 1, wherein the ketone is 4-hydroxyacetophenone.

6. The method of claim 1, wherein the aromatic hydroxy compound and the ketone are at a molar ratio of 5-30:1.

7. The method of claim 1, wherein the aromatic hydroxy compound and the ketone are at a molar ratio of 8-15:1.

8. (canceled)

9. (canceled)

10. The method of claim 1, wherein said mercaptan co-catalyst has a formula of:
[B [symbol] ₘ [symbol] A [symbol] [symbol] C]
wherein A is a monovalent or divalent hydrocarbyl group having 1 to 12 carbon atoms, B is selected from the group consisting of an hydrogen, a hydroxyl, —S—H, —S—R³, —COOR⁴ and SO₃R⁴, C is selected from the group consisting of —S—H, —S—R³, —SCOOR⁴ and SCOR⁴, wherein R³ is a tertiary alkyl group having 4 to 25 carbon atoms and R⁴ is selected from the group consisting of an hydrogen and a monovalent hydrocarbyl group having 1 to 12 carbon atoms, and m is an integer having a value 0 or 1.

11. The method of claim 1, wherein the mercaptan co-catalyst is 3-mercapto propionic acid.

12. (canceled)

13. (canceled)

14. The method of claim 1, further comprising reacting the 1,1,1-tris(4-hydroxyphenyl)alkane with one or more comonomers to form a branched polymer.

15. A method for forming 1,1,1-tris(4-hydroxyphenyl)ethane, comprising:
reacting a mixture of a phenol and a 2,4-pentanedione in the presence of at least one organic sulfonic acid catalyst and a mercaptan co-catalyst to form the 1,1,1-tris(4-hydroxyphenyl)ethane, wherein the organic sulfonic acid is in an amount from 4 to 8 weight percent based on a total weight of the mixture and wherein the mercaptan co-catalyst is in an amount from 0.75 weight percent to 3 weight percent based on the total weight of the mixture.

16. A method for forming 1,1,1-tris(4-hydroxyphenyl)ethane, comprising:
reacting a mixture of phenol and 4-hydroxyacetophenone in the presence of at least one organic sulfonic acid catalyst and a mercaptan co-catalyst to form the 1,1,1-tris(4-hydroxyphenyl)ethane, wherein the organic sulfonic acid is in an amount from 4 to 8 weight percent based on a total weight of the mixture and wherein the mercaptan co-catalyst is in an amount from 0.75 weight percent to 3 weight percent based on the total weight of the mixture.

17. A method comprising:
reacting a mixture comprising an aromatic hydroxy compound and a ketone in the presence of at least one organic sulfonic acid catalyst and a mercaptan co-catalyst, wherein the aromatic hydroxy compound has a formula of: 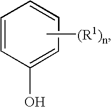
wherein the ketone has a formula of: 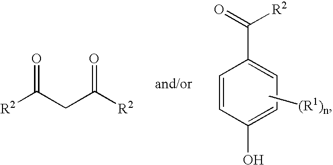
wherein R¹ and R² are independently at each occurrence a hydrocarbyl group and n is an integer of value 0-3, wherein the organic sulfonic acid is in an amount from 4 to 8 weight percent based on a total weight of the mixture and wherein the mercaptan co-catalyst is in an amount from 0.75 weight percent to 3 weight percent based on the total weight of the mixture;
contacting the mixture with a solvent to precipitate and isolate a filtrate and a 1,1,1-tris(4-hydroxyphenyl)alkane of formula: 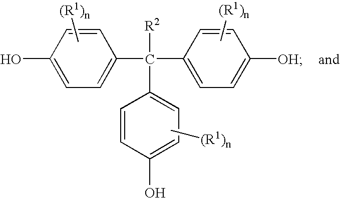
selectively removing the solvent from the filtrate to obtain a residue comprising the sulfonic acid catalyst and the mercaptan co-catalyst.

18. The method of claim 17, further comprising reacting the aromatic hydroxy compound and the ketone in the presence of the residue to form the 1,1,1-tris(4-hydroxyphenyl)alkane.
